(19) United States
(12) Patent Application Publication
(10) Pub. No.: US 2023/0226124 A1
(43) Pub. Date: Jul. 20, 2023

(54) METHOD FOR ANTI-AGEING BY USING PARABACTEROIDES GOLDSTEINII AND ITS GLYCOLIPID

(71) Applicant: Multistars Biotechnology Company Limited, Taoyuan City (TW)

(72) Inventors: Po-I Wu, Taoyuan City (TW); Hsin-Chih Lai, Taoyuan City (TW); Chia-Chen Lu, Taoyuan City (TW); Tzu-Lung Lin, Taoyuan City (TW)

(21) Appl. No.: 18/097,515

(22) Filed: Jan. 17, 2023

Related U.S. Application Data
(60) Provisional application No. 63/300,273, filed on Jan. 18, 2022.

(51) Int. Cl. A61K 35/74; A61Q 19/08

(52) U.S. Cl. CPC A61K 35/74; A61Q 19/08

(57) ABSTRACT

The present disclosure provides a method for anti-ageing by using Parabacteroides goldsteinii and its glycolipid. The Parabacteroides goldsteinii and its glycolipid of the present disclosure achieve anti-ageing effects through various efficacy experiments.

### CROSS-REFERENCE TO RELATED APPLICATION

This application claims priority of Provisional Application No. 63/300,273, filed on Jan. 18, 2022, the content of which is incorporated herein in its entirety by reference.

### BACKGROUND OF THE INVENTION

### 1. Field of the Invention

The present invention relates to a method for anti-ageing by using Parabacteroides goldsteinii and its glycolipid.

### 2. The Prior Art

Since ancient times, human beings have always dreamed of pursuing the way of eternal youth and immortality. In recent years, with the development and progress of medicine and biotechnology, not only can the front-end medical technology be used to fight against diseases, but many products that appeal to anti-ageing have also been developed and released one after another. In recent years, the trend of anti-ageing has slowly spread to the world. Taking Japan as an example, more than 70% of the people have anti-ageing awareness, and the concern of Taiwanese people for anti-ageing continues to increase. This anti-ageing trend will drive the market sales of anti-ageing related products, and the global market is expected to continue to expand.

Ageing phenomenon is a universal, progressive, cumulative and harmful physiological decline, but it cannot be described by a single or simple model based on individual differences. However, with the progress of research, it has been found that human ageing is closely related to reactive oxygen species (ROS) in the body. ROS can cause damage to DNA, protein, lipid and other molecules in cells in the body, resulting in the gradual loss of physiological functions, resulting in diseases and organ ageing. Many literatures point out that ROS is one of the main factors causing the ageing of internal organs. Therefore, the study of new anti-ageing products is one of the main topics in the field of anti-ageing research at home and abroad.

In view of the fact that current anti-ageing drugs or health foods still have disadvantages of side effects, cytotoxicity, chemical synthesis and poor effects. In order to solve the above-mentioned problems, those skilled in the art urgently need to develop a novel and effective method and pharmaceutical composition for anti-ageing for the benefit of a large group of people in need thereof.

### SUMMARY OF THE INVENTION

In order to solve the foregoing problems, an objective of the present invention is to provide a method for anti-ageing, comprising administering to a subject in need thereof a pharmaceutical composition comprising an effective amount of Parabacteroides goldsteinii and its glycolipid.

According to an embodiment of the present invention, the Parabacteroides goldsteinii is a live bacterium.

According to an embodiment of the present invention, the effective amount of Parabacteroides goldsteinii is at least 4×10⁹ CFUs per day for the subject.

According to an embodiment of the present invention, the effective amount of the glycolipid is at least 2 μg/0.1 ml per day for the subject.

According to an embodiment of the present invention, the Parabacteroides goldsteinii and its glycolipid reduce average escape latency of the subject.

According to an embodiment of the present invention, the Parabacteroides goldsteinii and its glycolipid improve locomotor activity deterioration of the subject.

According to an embodiment of the present invention, the Parabacteroides goldsteinii and its glycolipid improve body weight loss in the subject.

According to an embodiment of the present invention, the Parabacteroides goldsteinii and its glycolipid enhance gastrocnemius muscle and soleus muscle mass and weight in the subject.

According to an embodiment of the present invention, the Parabacteroides goldsteinii and its glycolipid reduce liver weight in the subject.

According to an embodiment of the present invention, the Parabacteroides goldsteinii and its glycolipid reduce sera glutamate pyruvate transaminase (GPT) and blood sugar levels in the subject.

According to an embodiment of the present invention, the Parabacteroides goldsteinii and its glycolipid reduce levels of sera Toll-like receptor 2 (TLR2) and Toll-like receptor 4 (TLR4) in the subject.

According to an embodiment of the present invention, the Parabacteroides goldsteinii and its glycolipid enhance running endurance in the subject.

According to an embodiment of the present invention, the Parabacteroides goldsteinii and its glycolipid enhance limb grip strength in the subject.

According to an embodiment of the present invention, the Parabacteroides goldsteinii and its glycolipid reduce average oxygen consumption (average VO₂), average carbon dioxide production (average VCO₂) and average respiratory exchange ratio (average RER) in the subject.

According to an embodiment of the present invention, the Parabacteroides goldsteinii and its glycolipid reduce oxygen consumption (VO₂) and carbon dioxide production (VCO₂) under light and dark condition in the subject.

According to an embodiment of the present invention, the Parabacteroides goldsteinii and its glycolipid increase respiratory exchange ratio (RER) under light condition and reduce the RER under dark condition in the subject.

According to an embodiment of the present invention, the pharmaceutical composition is in a dosage form for oral administration.

According to an embodiment of the present invention, the pharmaceutical composition is in a dosage form for parenteral administration.

According to an embodiment of the present invention, the pharmaceutical composition further comprises a pharmaceutically acceptable excipient, carrier, adjuvant and/or food additive.

In summary, the Parabacteroides goldsteinii and its glycolipid achieve anti-ageing effects through improving locomotor activity deterioration of the ageing subject, reducing average escape latency of the ageing subject, improving body weight loss in the ageing subject, enhancing gastrocnemius muscle and soleus muscle mass and weight in the ageing subject, reducing liver weight in the ageing subject, reducing sera glutamate pyruvate transaminase (GPT) and blood sugar levels in the ageing subject, reducing levels of sera Toll-like receptor 2 (TLR2) and Toll-like receptor 4 (TLR4) in the ageing subject, enhancing running endurance in the ageing subject, enhancing limb grip strength in the ageing subject, reducing average oxygen consumption (average VO₂), average carbon dioxide production (average VCO₂) and average respiratory exchange ratio (average RER) in the ageing subject, reducing oxygen consumption (VO₂) and carbon dioxide production (VCO₂) under light and dark condition in the ageing subject, increasing respiratory exchange ratio (RER) under light condition and reducing the RER under dark condition in the ageing subject.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows that Parabacteroides goldsteinii (Pg) strain MTS01 treatment in aged rats reduces the average escape latency. Old rats aged 26 months treated with and without Pg were subject to spatial learning and memory test of the Morris water maze test. Data representing means±standard deviation (SD) were analyzed using the unpaired t-test analysis. n=5 for each rats group. **, P<0.01. CTL represents control.

FIG. 2 shows that treatment of Parabacteroides goldsteinii strain MTS01 derived glycolipid (i.e., Pg-GLC) in aged rats reduces the average escape latency. Old rats aged 26 months treated with and without Pg-GLC treatment were subject to spatial learning and memory test of the Morris water maze test. Data representing means±SD were analyzed using the unpaired t-test analysis. n=5 for each rats group. *, P<0.05. CTL represents control.

FIG. 3 shows that orally administration of Parabacteroides goldsteinii (Pg) strain MTS01 improves the locomotor activity deterioration of ageing rats. Pg treatment in aged rats increases the total activity (distance, mm) in contrast to the control (CTL) group. Data representing means±SD were analyzed using the unpaired t-test analysis. n=5 for each rats group. ***, P<0.001. CTL represents control.

FIG. 4 shows that Pg-GLC treatment by intraperitoneal injection improves the locomotor activity deterioration of ageing rats. Pg-GLC treatment in aged rats increases the total activity (distance, mm) in contrast to the control (CTL) group. Data representing means±SD were analyzed using the unpaired t-test analysis. n=5 for each rats group. *, P<0.05. CTL represents control.

FIGS. 5A and 5B show effect of administration of Parabacteroides goldsteinii (Pg) strain MTS01 on body weight change in ageing mice. (FIG. 5A) Aged: aged mice treated with 0.2 ml PBS. Young: young mice treated with 0.2 ml PBS. Aged C: aged mice treated with 0.2 ml PBS containing creatine (1.5 mg/mouse/day). Aged PG: aged mice treated with live Parabacteroides goldsteinii strain MTS01 at 5×10⁸ C.F.U./0.2 ml PBS. (FIG. 5B) Results in FIG. 5A was quantified and shown in FIG. 5B. Aged mice were at 83 weeks old when study initiated. Young mice were at 8 weeks old when study initiated. All mice were treated with 0.2 ml PBS, five days a week for four weeks. *: P<0.05; ****: P<0.001. NS: not significant.

FIG. 6 shows effect of administration of Parabacteroides goldsteinii (Pg) strain MTS01 on gastrocnemius muscle and soleus muscle mass in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. GA: gastrocnemius muscle; SOL: soleus muscle.

FIG. 7 shows Effect of administration of Parabacteroides goldsteinii (Pg) strain MTS01 on soleus muscle mass in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. Muscle was subject to hematoxylin and eosin (HE) staining.

FIG. 8 shows effect of administration of Parabacteroides goldsteinii (Pg) strain MTS01 on weight of gastrocnemius muscle, soleus muscle and liver in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. *: P<0.05; **: P<0.01; ****: P<0.001. NS: not significant.

FIG. 9 shows effect of administration of Parabacteroides goldsteinii (Pg) strain MTS01 on sera glutamate pyruvate transaminase (GPT) and blood sugar levels in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. GPT: glutamate pyruvate transaminase. *: P<0.05; **: P<0.01. NS: not significant.

FIG. 10 shows effect of administration of Parabacteroides goldsteinii (Pg) strain MTS01 on levels of sera Toll-like receptor 2 (TLR2) and Toll-like receptor 4 (TLR4) activities in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. TLR2: Toll-like receptor 2; TLR4: Toll-like receptor 4. *: P<0.05. NS: not significant.

FIG. 11 shows effect of administration of Parabacteroides goldsteinii (Pg) strain MTS01 on running endurance in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. 1st 2^{nd} and 3^{rd} meant the times of test. *: P<0.05. **: P<0.01; ***: P<0.005; ****: P<0.001. NS: not significant. The running endurance of the mice was evaluated by the rotarod test.

FIG. 12 shows effect of administration of Parabacteroides goldsteinii (Pg) strain MTS01 on grip strength in the forelimb in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. 1^{st}, 2^{nd}, 3^{rd}, 4^{th} and 5^{th} meant the times of test. *: P<0.05. **: P<0.01; ****: P<0.001. NS: not significant.

FIG. 13 shows effect of administration of Parabacteroides goldsteinii (Pg) strain MTS01 on metabolic cart in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. *: P<0.05. **: P<0.01. NS: not significant.

FIG. 14 shows effect of administration of Parabacteroides goldsteinii (Pg) strain MTS01 on metabolic cart under light and dark condition in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. *: P<0.05. **: P<0.01. ***: P<0.005: NS: not significant.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

As used herein, the data provided represent experimental values that can vary within a range of ±20%, preferably within ±10%, and most preferably within ±5%.

As used herein, the term “effective dose” refers to the amount of Parabacteroides goldsteinii required for anti-ageing. The effective dose may be different depending on the biological species or individual differences to be treated, but the effective dose can be determined experimentally by, for example, dose escalation.

According to the present invention, the operating procedures and parameter conditions related to bacterial culture fall within the scope of the professional literacy and routine techniques of those skilled in the art.

As used herein, the term “bacterial component” refers to the derivative substances directly or indirectly related to the bacteria when the bacteria are cultivated, including but not limited to the metabolites of the bacteria, the structure of the bacteria, active and inactive components related to the bacteria, etc.

According to the present invention, the pharmaceutical composition can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, and transdermal administration.

According to the present invention, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

Aged rats often display impairments in long-term spatial memory and novel-object recognition. The Morris water maze is a convenient and standard test of cognitive function in rodents. Morris water maze from the behavior test was also used for the analysis of effect of ageing in retention of learned spatial location over short- and long-term intervals. We first investigated the effects of Parabacteroides goldsteinii (Pg) or its derived glycolipid, Pg-GLC on acquisition and reference memory in the Morris water maze in old rats aged 26-month.

### Example 1

Parabacteroides goldsteinii Strain MTS01 Cultivation, Isolation of Pg-GLC and Evaluation of Effect of Parabacteroides goldsteinii Strain MTS01 and Pg-GLC on Reducing Average Escape Latency of Ageing Subject and Improving Locomotor Activity Deterioration of Ageing Subject

The cultivation procedure of Parabacteroides goldsteinii strain MTS01 is as follows. The Parabacteroides goldsteinii strain MTS01 was deposited in the Deutsche Sammlung von Mikroorganismen and Zellkulturen (DSMZ) on Oct. 29, 2018, under accession number DSM 32939. The Parabacteroides goldsteinii strain MTS01 in the present application is a strain that is easily obtained by those ordinarily skilled in the art, and has been disclosed in U.S. application Ser. No. 16/541,308 (corresponding to U.S. Pat. No. 11,166,988). Parabacteroides goldsteinii strain MTS01 was cultivated on anaerobic blood agar (Creative, New Taipei city, Taiwan) and liquid thioglycollate medium (BD, Franklin Lakes, USA) at 37° C. in a Whitley DG250 anaerobic chamber (Don Whitley, Bingley, UK) with mixed anaerobic gas (5% carbon dioxide, 5% hydrogen, 90% nitrogen). Anaerobic condition was confirmed using an anaerobic indicator (Oxoid, Hampshire, UK).

The operating procedure of the animal experiment is as follows. Sprague-Dawley (SD) rats aged 26 months were used for test and kept under sterile conditions (following a 12 h light/dark cycle), with one-week acclimatization period. Aged SD rats were treated with 0.1 ml 0.9% normal saline, or with Parabacteroides goldsteinii (4×10⁹ CFU per day) or with Pg-GLC [intraperitoneal (i.p.) injection; 2 μg/0.1 ml per day].

The isolation procedure of Pg-GLC is as follows. The glycolipid purified from Parabacteroides goldsteinii strain MTS01 (Pg-GLC) was used for test. The glycolipid was isolated by using the hot phenol-water extraction. Bacterial pellet of 1200 ml overnight culture were suspended in 30 ml of the warm water and added an equal volume of phenol, then stirred at 65° C. for 30 minutes. After centrifugation, the aqueous layers were collected, and the organic layer were added an equal volume of warm water to perform the extraction twice. The crude extract obtained after dialysis and lyophilization were treated with DNase, RNase and Proteinase K. Further dialysis and lyophilization were performed to obtain the purified glycolipid.

The experimental procedure of the Morris water maze behavioral test is as follows. A Morris water maze system was used for behavioral training and testing. The training and testing included two stages, i.e., “navigation test” and “experimental space exploration”. The navigation test was done on a platform placed in the fourth quadrant of a round pool. The rats were placed into the pool facing any point on the wall. A video system automatically recorded the swimming paths of the rats and exactly counted the time for each rat to find the platform. After a rat found the platform, or if a rat failed to find the platform within 2 min (the time limit to consider as latency), the rat was guided to the platform and allowed to rest for 10-20 s, after which the next experiment was performed. Rats underwent training 4 times daily, for five days, and all training sessions were performed in the darkness. Subsequently, the space exploration experiments were done 24 h after the navigation test ended. The platform was removed, and the rat was placed in the second quadrant of the pool. The time needed for the rats swam through the area where the platform originally located (i.e., the fourth quadrant) was recorded and the distance of swimming in the fourth quadrant versus the total distance was calculated (see Charles V Vorhees and Michael T Williams. Morris water maze procedures for assessing spatial and related forms of learning and memory. Nat Protoc. 2006; 1(2): 848-858.)

The experimental procedure of spatial learning and memory performance increasing in Pg-treated old rats is as follows. To examine the effect of Pg in spatial learning and memory of old rats, behavioral data of the Morris water maze test were performed.

The experimental procedure of the anxiety-like behavior is as follows. Prevalence of emotional distress, including high-trait anxiety and depression, is a significant public health problem in the elderly (see Thomas M. Richardson, Adam Simning, Hua He, Yeates Conwell. Anxiety and its correlates among older adults accessing ageing services. Int J Geriatr Psychiatry. 2011 January; 26(1): 31-38.) Estimates of the prevalence of anxiety in old age varies widely from 1.2-14 to 1-28% in community versus clinical contexts, respectively (see Olivia Remes, Carol Brayne, Rianne van der Linde, Louise Lafortune. A systematic review of reviews on the prevalence of anxiety disorders in adult populations. Brain Behay. 2016 July; 6(7): e00497.) Prevalence is even higher in older people with mild cognitive impairment, ranging from 11 to 75% (see Roberto Monastero, Francesca Mangialasche, Cecilia Camarda, Rosolino Camarda. A systematic review of neuropsychiatric symptoms in mild cognitive impairment. Journal of Alzheimer's disease: JAD. 2009 July; 18(1):11-30; Jen Yates, Linda Clare, Bob Woods. Mild cognitive impairment and mood: A systematic review. Reviews in Clinical Gerontology. 2013 November; 23(04).) The corresponding behavioral trait of anxiety-like behavior in rodents was examined in the open field test as shown below (see Michael L. Seibenhener and Michael C. Wooten. Use of the Open Field Maze to Measure Locomotor and Anxiety-like Behavior in Mice. J Vis Exp. 2015; (96): 52434.)

The experimental procedure of the open field test is as follows. The open field test was used to estimate anxiety-like behavior in a novel environment (see Walsh R N and Cummins R A. The open field test—a critical review. Psychol Bull. 1976; 83: 482-504.) For analysis, the open field was divided into virtual 25 squares; the central nine squares were defined as the center of the open field, and the 16 squares along the walls of the field were defined as the periphery. Over a test period of 15 min, total time spent in the periphery of the field and total immobility time, regardless of location, was measured for each mouse. Open field test is widely used to measure locomotor activity and anxiety-like behavior. The older age groups tended to exhibit reduction in vertical activity and center time compared with the younger age groups (see Hirotaka Shoji, Keizo Takao, Satoko Hattori, Tsuyoshi Miyakawa. Age-related changes in behavior in C57BL/6J mice from young adulthood to middle age. Shoji et al. Molecular Brain. 2016 December; 9:11.)

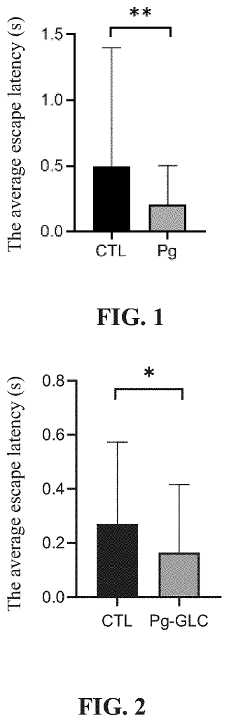

FIG. 1 shows that Parabacteroides goldsteinii (Pg) strain MTS01 treatment in aged rats reduces the average escape latency. Old rats aged 26 months treated with and without Pg were subject to spatial learning and memory test of the Morris water maze test. Data representing means±standard deviation (SD) were analyzed using the unpaired t-test analysis. n=5 for each rats group. **, P<0.01. CTL represents control.

FIG. 2 shows that treatment of Parabacteroides goldsteinii strain MTS01 derived glycolipid (i.e., Pg-GLC) in aged rats reduces the average escape latency. Old rats aged 26 months treated with and without Pg-GLC treatment were subject to spatial learning and memory test of the Morris water maze test. Data representing means±SD were analyzed using the unpaired t-test analysis. n=5 for each rats group. *, P<0.05. CTL represents control.

As shown in FIG. 1, the average escape latency, expressed as seconds (s) of aged rats significantly decreased in Pg-treated group, compared with that of CTL group. As shown in FIG. 2, similar beneficial effects that Pg-GLC treatment significantly improves the spatial learning and memory of old rats were also observed. In general, enhanced locomotor activity and improved anxiety-like behaviors in Pg- or Pg-GLC treated old SD rats were observed.

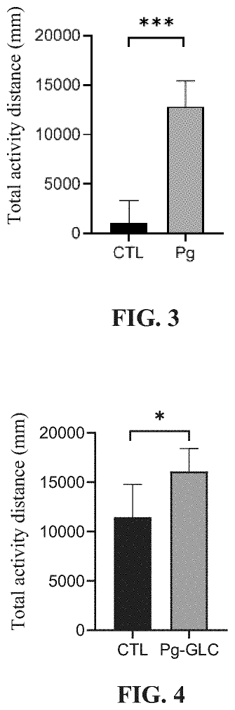

FIG. 3 shows that orally administration of Parabacteroides goldsteinii (Pg) strain MTS01 improves the locomotor activity deterioration of ageing rats. Pg treatment in aged rats increases the total activity (distance, mm) in contrast to the control (CTL) group. Data representing means±SD were analyzed using the unpaired t-test analysis. n=5 for each rats group. ***, P<0.001. CTL represents control.

During the first experimental period of time, aged SD rats were either treated with 0.1 ml 0.9% normal saline (CTL group), or with Pg (4×10⁹ CFU per day) (Pg group), respectively. After 1 month, the total traveled distance was significantly increased in Pg-treated rats compared with that of CTL group (see FIG. 3).

FIG. 4 shows that Pg-GLC treatment by intraperitoneal injection improves the locomotor activity deterioration of ageing rats. Pg-GLC treatment in aged rats increases the total activity (distance, mm) in contrast to the control (CTL) group. Data representing means±SD were analyzed using the unpaired t-test analysis. n=5 for each rats group. *, P<0.05. CTL represents control.

In the second experiment, aged SD rats were either treated with 0.1 ml 0.9% normal saline (CTL group), or with Pg-GLC [intraperitoneal (i.p.) injection; 2 μg/0.1 ml per day] (Pg-GLC group), respectively. After 1 month, the total traveled distance was significantly increased in Pg-GLC treated rats compared with that of CTL group (see FIG. 4).

Overall, Pg or Pg-GLC treatment improves that the locomotor activity deterioration of aged rats during the entire testing period in an open field environment. These results suggest that Pg or Pg-GLC ameliorates ageing-associated anxiety-like behavior.

### Example 2

Evaluation of Effect of Administration of Parabacteroides goldsteinii (Pg) Strain MTS01 on Improving Body Weight Loss in Ageing Subject

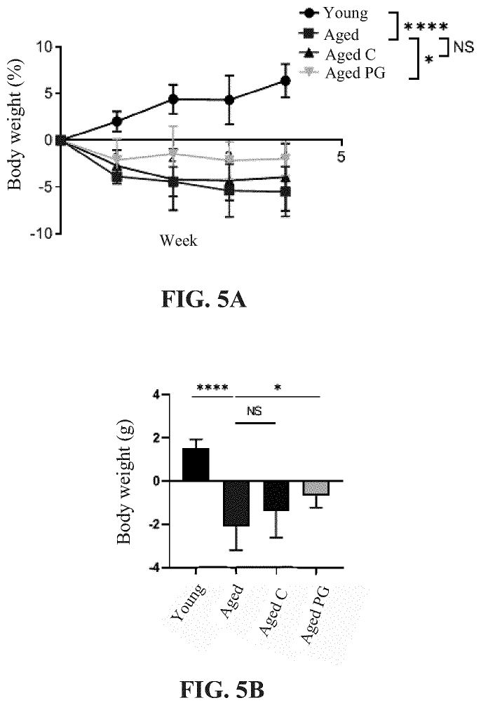

This example investigates the effect of administration of Parabacteroides goldsteinii (Pg) strain MTS01 on improving body weight loss in the ageing subject. The result is shown in FIGS. 5A and 5B.

FIGS. 5A and 5B show effect of administration of Parabacteroides goldsteinii (Pg) strain MTS01 on body weight change in ageing mice. (FIG. 5A) Aged: aged mice treated with 0.2 ml PBS. Young: young mice treated with 0.2 ml PBS. Aged C: aged mice treated with 0.2 ml PBS containing creatine (1.5 mg/mouse/day). Aged PG: aged mice treated with live Parabacteroides goldsteinii strain MTS01 at 5×10⁸ C.F.U./0.2 ml PBS. (FIG. 5B) Results in FIG. 5A was quantified and shown in FIG. 5B. Aged mice were at 83 weeks old when study initiated. Young mice were at 8 weeks old when study initiated. All mice were treated with 0.2 ml PBS, five days a week for four weeks. *: P<0.05; ****: P<0.001. NS: not significant.

The result of this example shows that Parabacteroides goldsteinii (Pg) strain MTS01 has the effect on improving body weight loss in the ageing subject.

### Example 3

Evaluation of Effect of Administration of Parabacteroides goldsteinii (Pg) Strain MTS01 on Enhancing Gastrocnemius Muscle and Soleus Muscle Mass and Weight in Ageing Subject, and Reducing Liver Weight in Ageing Subject

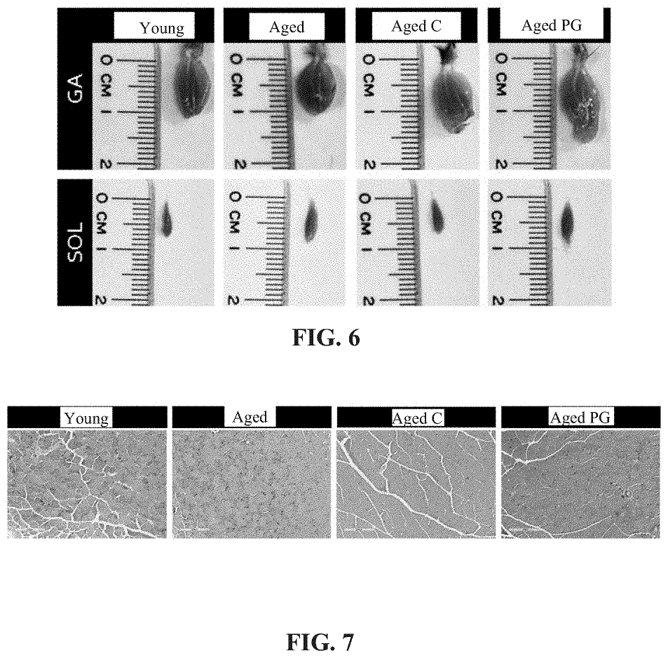

This example investigates the effect of administration of Parabacteroides goldsteinii (Pg) strain MTS01 on enhancing gastrocnemius muscle and soleus muscle mass and weight in the ageing subject, and reducing liver weight in the ageing subject. The result is shown in FIGS. 6-8.

FIG. 6 shows effect of administration of Parabacteroides goldsteinii (Pg) strain MTS01 on gastrocnemius muscle and soleus muscle mass in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. GA: gastrocnemius muscle; SOL: soleus muscle.

FIG. 7 shows Effect of administration of Parabacteroides goldsteinii (Pg) strain MTS01 on soleus muscle mass in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. Muscle was subject to hematoxylin and eosin (HE) staining.

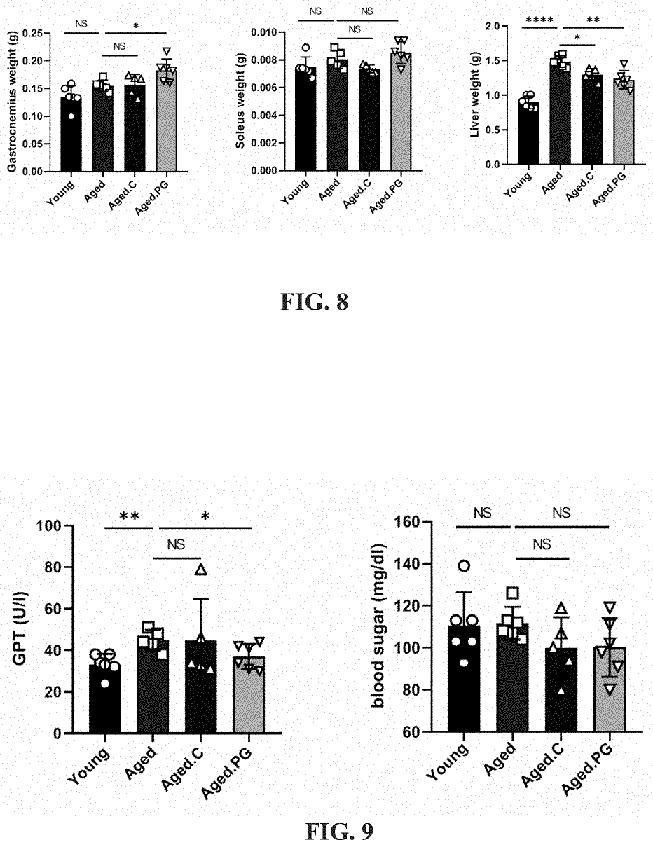

FIG. 8 shows effect of administration of Parabacteroides goldsteinii (Pg) strain MTS01 on weight of gastrocnemius muscle, soleus muscle and liver in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. *: P<0.05; **: P<0.01; ****: P<0.001. NS: not significant.

The result of this example shows that Parabacteroides goldsteinii (Pg) strain MTS01 has the effect on enhancing gastrocnemius muscle and soleus muscle mass and weight in the ageing subject, and reducing liver weight in the ageing subject.

### Example 4

Evaluation of Effect of Administration of Parabacteroides goldsteinii (Pg) Strain MTS01 on Reducing Sera Glutamate Pyruvate Transaminase (GPT) and Blood Sugar Levels in Ageing Subject

This example investigates the effect of administration of Parabacteroides goldsteinii (Pg) strain MTS01 on reducing sera glutamate pyruvate transaminase (GPT) and blood sugar levels in the ageing subject. The result is shown in FIG. 9.

FIG. 9 shows effect of administration of Parabacteroides goldsteinii (Pg) strain MTS01 on sera glutamate pyruvate transaminase (GPT) and blood sugar levels in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. GPT: glutamate pyruvate transaminase. *: P<0.05; **: P<0.01. NS: not significant.

The result of this example shows that Parabacteroides goldsteinii (Pg) strain MTS01 has the effect on reducing sera GPT and blood sugar levels in the ageing subject.

### Example 5

Evaluation of Effect of Administration of Parabacteroides goldsteinii (Pg) Strain MTS01 on Reducing Levels of Sera Toll-Like Receptor 2 (TLR2) and Toll-Like Receptor 4 (TLR4) in Ageing Subject

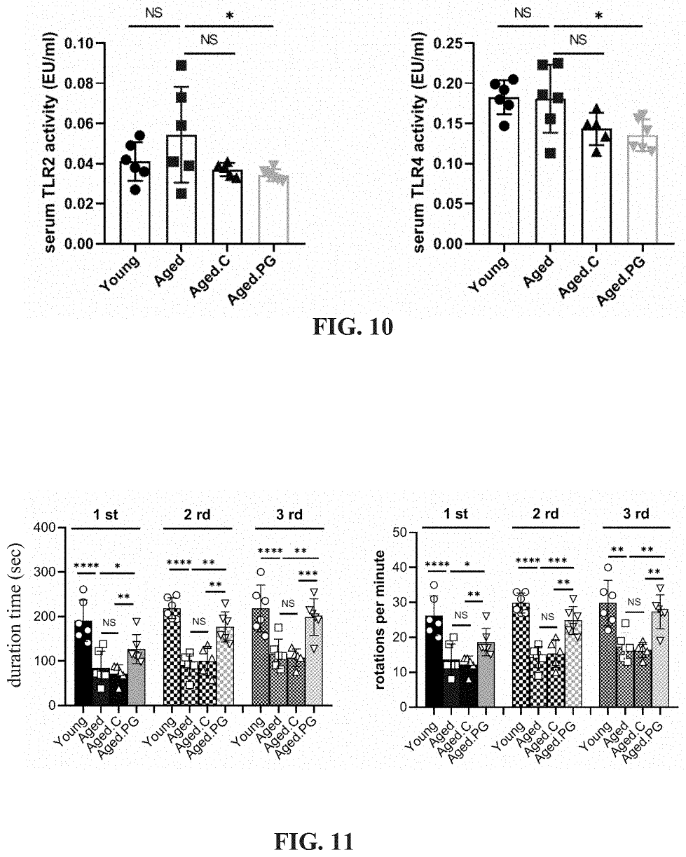

This example investigates the effect of administration of Parabacteroides goldsteinii (Pg) strain MTS01 on reducing levels of sera Toll-like receptor 2 (TLR2) and Toll-like receptor 4 (TLR4) in the ageing subject. The result is shown in FIG. 10.

FIG. 10 shows effect of administration of Parabacteroides goldsteinii (Pg) strain MTS01 on levels of sera Toll-like receptor 2 (TLR2) and Toll-like receptor 4 (TLR4) activities in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. TLR2: Toll-like receptor 2; TLR4: Toll-like receptor 4. *: P<0.05. NS: not significant.

The result of this example shows that Parabacteroides goldsteinii (Pg) strain MTS01 has the effect on reducing levels of sera TLR2 and TLR4 in the ageing subject.

### Example 6

Evaluation of Effect of Administration of Parabacteroides goldsteinii (Pg) Strain MTS01 on Enhancing Running Endurance in Ageing Subject

This example investigates the effect of administration of Parabacteroides goldsteinii (Pg) strain MTS01 on enhancing running endurance in the ageing subject. The result is shown in FIG. 11.

FIG. 11 shows effect of administration of Parabacteroides goldsteinii (Pg) strain MTS01 on running endurance in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. 1^{st}, 2^{nd}, and 3^{rd} meant the times of test. *: P<0.05. **: P<0.01; ***: P<0.005; ****: P<0.001. NS: not significant. The running endurance of the mice was evaluated by the rotarod test.

The result of this example shows that Parabacteroides goldsteinii (Pg) strain MTS01 has the effect on enhancing running endurance in the ageing subject.

### Example 7

Evaluation of Effect of Administration of Parabacteroides goldsteinii (Pg) Strain MTS01 on Enhancing Limb Grip Strength in Ageing Subject

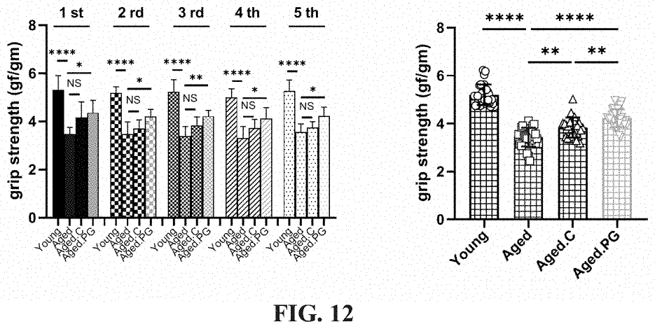

This example investigates the effect of administration of Parabacteroides goldsteinii (Pg) strain MTS01 on enhancing limb grip strength in the ageing subject. The result is shown in FIG. 12.

FIG. 12 shows effect of administration of Parabacteroides goldsteinii (Pg) strain MTS01 on grip strength in the forelimb in ageing mice. The grip strength of the limbs is examined by using the muromachi company model MK-380CM/R rat and mouse grip tester. The pulling force of the mouse's forelimb was calculated by detecting the force with which the mouse grasps the horizontal grip. Differences in grasping ability or muscle strength of mice can be obtained by measuring in this way. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. 1^{st}, 2^{nd}, 3^{rd}, 4^{th}, and 5^{th} meant the times of test. *: P<0.05. **: P<0.01; ****: P<0.001. NS: not significant.

The result of this example shows that Parabacteroides goldsteinii (Pg) strain MTS01 has the effect on enhancing limb grip strength in the ageing subject.

### Example 8

Evaluation of Effect of Administration of Parabacteroides goldsteinii (Pg) Strain MTS01 on Reducing Average Oxygen Consumption (Average VO₂), Average Carbon Dioxide Production (Average VCO₂) and Average Respiratory Exchange Ratio (Average RER) in Ageing Subject

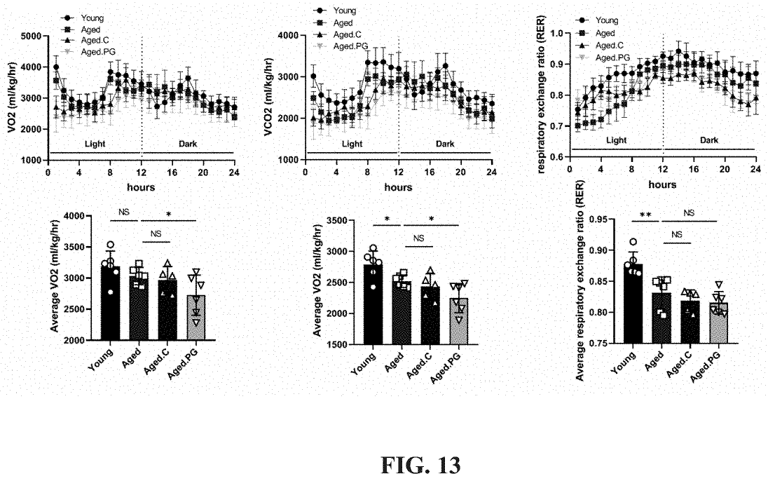

This example investigates the effect of administration of Parabacteroides goldsteinii (Pg) strain MTS01 on reducing average oxygen consumption (average VO₂), average carbon dioxide production (average VCO₂) and average respiratory exchange ratio (average RER) in the ageing subject. The result is shown in FIG. 13.

FIG. 13 shows effect of administration of Parabacteroides goldsteinii (Pg) strain MTS01 on metabolic cart in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. *: P<0.05. **: P<0.01. NS: not significant.

The result of this example shows that Parabacteroides goldsteinii (Pg) strain MTS01 has the effect on reducing average oxygen consumption (average VO₂), average carbon dioxide production (average VCO₂) and average respiratory exchange ratio (average RER) in the ageing subject.

### Example 9

Evaluation of Effect of Administration of Parabacteroides goldsteinii (Pg) Strain MTS01 on Reducing Oxygen Consumption (VO₂) and Carbon Dioxide Production (VCO₂) Under Light and Dark Condition in Ageing Subject, and Increasing Respiratory Exchange Ratio (RER) Under Light Condition and Reducing RER Under Dark Condition in Ageing Subject

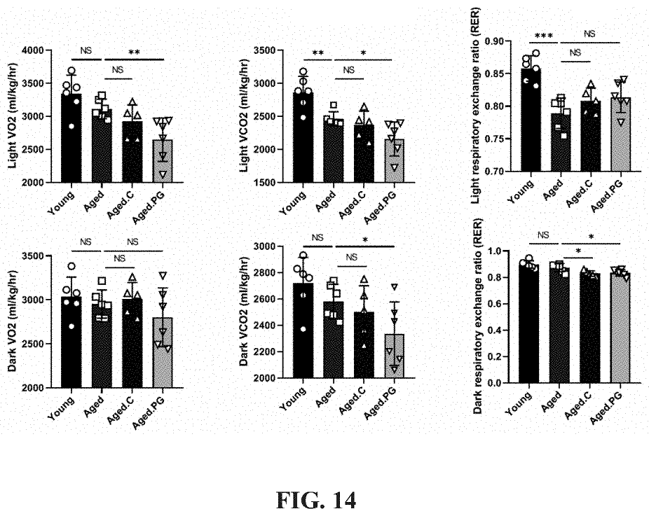

This example investigates the effect of administration of Parabacteroides goldsteinii (Pg) strain MTS01 on reducing oxygen consumption (VO₂) and carbon dioxide production (VCO₂) under light and dark condition in the ageing subject, and increasing respiratory exchange ratio (RER) under light condition and reducing RER under dark condition in the ageing subject. The result is shown in FIG. 14.

FIG. 14 shows effect of administration of Parabacteroides goldsteinii (Pg) strain MTS01 on metabolic cart under light and dark condition in ageing mice. Young: young mice treated with 0.2 ml PBS. Aged: aged mice treated with 0.2 ml PBS. Aged C: aged mice treated with 0.2 ml PBS containing creatine (1.5 mg/mouse/day). Aged PG: aged mice treated with live Parabacteroides goldsteinii (Pg) strain MTS01 at 5×10⁸ C.F.U./0.2 ml PBS. Aged mice were at 83 weeks old when study initiated. Young mice were at 8 weeks old when study initiated. All mice were treated with 0.2 ml PBS, five days a week for four weeks. *: P<0.05. **: P<0.01. ***: P<0.005: NS: not significant.

The result of this example shows that Parabacteroides goldsteinii (Pg) strain MTS01 has the effect on reducing oxygen consumption (VO₂) and carbon dioxide production (VCO₂) under light and dark condition in the ageing subject, and increasing respiratory exchange ratio (RER) under light condition and reducing RER under dark condition in the ageing subject.

In summary, the Parabacteroides goldsteinii and its glycolipid achieve anti-ageing effects through improving locomotor activity deterioration of the ageing subject, reducing average escape latency of the ageing subject, improving body weight loss in the ageing subject, enhancing gastrocnemius muscle and soleus muscle mass and weight in the ageing subject, reducing liver weight in the ageing subject, reducing sera glutamate pyruvate transaminase (GPT) and blood sugar levels in the ageing subject, reducing levels of sera Toll-like receptor 2 (TLR2) and Toll-like receptor 4 (TLR4) in the ageing subject, enhancing running endurance in the ageing subject, enhancing limb grip strength in the ageing subject, reducing average oxygen consumption (average VO₂), average carbon dioxide production (average VCO₂) and average respiratory exchange ratio (average RER) in the ageing subject, reducing oxygen consumption (VO₂) and carbon dioxide production (VCO₂) under light and dark condition in the ageing subject, increasing respiratory exchange ratio (RER) under light condition and reducing the RER under dark condition in the ageing subject.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for anti-ageing, comprising administering to a subject in need thereof a pharmaceutical composition comprising an effective amount of Parabacteroides goldsteinii and its glycolipid.

2. The method according to claim 1, wherein the Parabacteroides goldsteinii is a live bacterium.

3. The method according to claim 1, wherein the effective amount of Parabacteroides goldsteinii is at least 4×10⁹ CFUs per day for the subject.

4. The method according to claim 1, wherein the effective amount of the glycolipid is at least 2 μg/0.1 ml per day for the subject.

5. The method according to claim 1, wherein the Parabacteroides goldsteinii and its glycolipid reduce average escape latency of the subject.

6. The method according to claim 1, wherein the Parabacteroides goldsteinii and its glycolipid improve locomotor activity deterioration of the subject.

7. The method according to claim 1, wherein the Parabacteroides goldsteinii and its glycolipid improve body weight loss in the subject.

8. The method according to claim 1, wherein the Parabacteroides goldsteinii and its glycolipid enhance gastrocnemius muscle and soleus muscle mass and weight in the subject.

9. The method according to claim 1, wherein the Parabacteroides goldsteinii and its glycolipid reduce liver weight in the subject.

10. The method according to claim 1, wherein the Parabacteroides goldsteinii and its glycolipid reduce sera glutamate pyruvate transaminase (GPT) and blood sugar levels in the subject.

11. The method according to claim 1, wherein the Parabacteroides goldsteinii and its glycolipid reduce levels of sera Toll-like receptor 2 (TLR2) and Toll-like receptor 4 (TLR4) in the subject.

12. The method according to claim 1, wherein the Parabacteroides goldsteinii and its glycolipid enhance running endurance in the subject.

13. The method according to claim 1, wherein the Parabacteroides goldsteinii and its glycolipid enhance limb grip strength in the subject.

14. The method according to claim 1, wherein the Parabacteroides goldsteinii and its glycolipid reduce average oxygen consumption (average VO₂), average carbon dioxide production (average VCO₂) and average respiratory exchange ratio (average RER) in the subject.

15. The method according to claim 14, wherein the Parabacteroides goldsteinii and its glycolipid reduce oxygen consumption (VO₂) and carbon dioxide production (VCO₂) under light and dark condition in the subject.

16. The method according to claim 14, wherein the Parabacteroides goldsteinii and its glycolipid increase respiratory exchange ratio (RER) under light condition and reduce the RER under dark condition in the subject.

17. The method according to claim 1, wherein the pharmaceutical composition is in a dosage form for oral administration.

18. The method according to claim 1, wherein the pharmaceutical composition is in a dosage form for parenteral administration.

19. The method according to claim 1, wherein the pharmaceutical composition further comprises a pharmaceutically acceptable excipient, carrier, adjuvant and/or food additive.
